(19) United States
(12) Patent Application Publication
(10) Pub. No.: US 2025/0186738 A1
(43) Pub. Date: Jun. 12, 2025

(54) MEDICAL DEVICES WITH RADIOPAQUE COILS

(71) Applicants: STRYKER CORPORATION, Kalamazoo, MI (US); STRYKER EUROPEAN OPERATIONS LIMITED, Carrigtwohill (IE)

(72) Inventors: Clay William Northrop, Salt Lake City, UT (US); Ted William Layman, Park City, UT (US)

(73) Assignees: STRYKER CORPORATION, Kalamazoo, MI (US); STRYKER EUROPEAN OPERATIONS LIMITED, Carrigtwohill (IE)

(21) Appl. No.: 19/054,595

(22) Filed: Feb. 14, 2025

Related U.S. Application Data
(63) Continuation of application No. PCT/US2023/070586, filed on Jul. 20, 2023.
(60) Provisional application No. 63/401,263, filed on Aug. 26, 2022.

(51) Int. Cl. A61M 25/00; A61M 25/09

(52) U.S. Cl. CPC A61M 25/005; A61M 25/0068; A61M 25/09; A61M 2025/09091; A61M 2025/09166; A61M 2025/09175; A61M 2205/0216; A61M 2205/0233; A61M 2205/025; A61M 2205/32

(57) ABSTRACT

A medical device includes: an elongate member having a proximal end, a distal end, and a body extending from the proximal end to the distal end; a blunt tip at or coupled to the distal end of the elongate member; and a radiopaque coil coupled to the elongate member; wherein the radiopaque coil is shapable and has a shape-retention characteristic, and wherein the shapable radiopaque coil with the shape-retention characteristic has a bending stiffness that is at least 10% of a bending stiffness of the medical device.

### RELATED APPLICATION DATA

This application is a continuation of International Patent Application No. PCT/US2023/070586, filed Jul. 20, 2023, which claims priority to U.S. Provisional Patent Application No. 63/401,263, filed Aug. 26, 2022, the disclosures of all of which are hereby incorporated herein by reference in their entirety into the present application.

### FIELD

The field of the application relates to medical devices, and more specifically, to guidewires and catheters.

### BACKGROUND

Guidewires have been used in the medical field to access passages inside patients. In some cases, it may be desirable for a guidewire to have good torqueability, which allows a torque motion applied about a longitudinal axis of the guidewire at a proximal end of the guidewire to cause a corresponding twisting motion at a distal end of the guidewire.

Also, it may be desirable for a distal segment of a guidewire to retain a certain bent shape during use. This allows the distal segment of the guidewire to access certain passage with specific geometry inside the patient. If the distal segment of the guidewire cannot retain its bent shape during use, then it may not be able to access a target passage.

In addition, it may be desirable for a guidewire to have a soft distal segment. This prevents the guidewire from causing injury to the patient, and also allows the guidewire to elastically flex or bend as it is advanced inside the patient through passages of different shapes.

However, it is difficult for a guidewire to achieve all of the above desirable features. A guidewire may have a soft distal segment, but such guidewire may have poor shape retention ability at the distal segment and poor torqueability. On the other hand, a guidewire may have great shape retention ability at the distal segment and good torqueability. However, such guidewire may have a stiff distal segment. The above desirable features are difficult to accomplish together because a soft distal segment of a guidewire usually cannot achieve good torqueability due to the softness of the material that is used to make the distal segment. Also, the material that is used to make the soft distal guidewire segment may not allow the distal guidewire segment to maintain its shape during use.

One or more of the above technical challenges also similarly apply for catheters.

### SUMMARY

A medical device includes: an elongate member having a proximal end, a distal end, and a body extending from the proximal end to the distal end; a blunt tip at or coupled to the distal end of the elongate member; and a radiopaque coil coupled to the elongate member; wherein the radiopaque coil is shapable and has a shape-retention characteristic, and wherein the shapable radiopaque coil with the shape-retention characteristic has a bending stiffness that is at least 10% of a bending stiffness of the medical device.

Optionally, the bending stiffness of the shapable radiopaque coil is at least 20% of the bending stiffness of the medical device.

Optionally, the shapable radiopaque coil with the shape-retention characteristic is made from a material having an elastic modulus that is at least 3E7 psi.

Optionally, the shapable radiopaque coil with the shape-retention characteristic is made from Molybdenum Rhenium (MoRe), or another Molybdenum alloy, or from Tungsten, Tungsten Rhenium (WRe), or another Tungsten alloy.

Optionally, the radiopaque coil is configured to assist retention of a shape for the medical device.

Optionally, the bending stiffness of the radiopaque coil is greater than a bending stiffness of a coil having a same size and shape as those of the radiopaque coil, but is made from platinum or platinum alloys.

Optionally, the radiopaque coil has been heat-treated to optimize a shape-ability and the shape retention characteristic of the radiopaque coil.

Optionally, the medical device is a guidewire.

Optionally, the elongate member is a shaft, and wherein the radiopaque coil surrounds at least a part of the shaft. Optionally, the medical device further includes a sleeve disposed around the radiopaque coil.

Optionally, the shaft comprises a flat portion. Optionally, the shaft comprises a tapering portion that is proximal the flat portion.

Optionally, the shaft comprises an additional flat portion or a cylinder portion that is proximal the tapering portion.

Optionally, the medical device is a catheter.

Optionally, the elongate member is a tubular member, and wherein the radiopaque coil is coupled to the tubular member.

Optionally, the radiopaque coil is disposed circumferentially around the tubular member, or is distal to the tubular member.

Optionally, the blunt tip comprises a rectilinear surface at the distal end of the elongate member.

A guidewire includes: a shaft having a proximal end, a distal end, and a body extending from the proximal end to the distal end; a blunt tip coupled to the distal end of the shaft; and a radiopaque coil disposed around at least a part of the shaft; wherein the radiopaque coil is shapable and has a shape-retention characteristic; and wherein the shapable radiopaque coil with the shape-retention characteristic has a bending stiffness that is at least 10% of a bending stiffness of the guidewire.

A catheter includes: a tubular member having a proximal end, a distal end, and a body extending from the proximal end to the distal end; and a radiopaque coil coupled to the tubular member; wherein the radiopaque coil is shapable and has a shape-retention characteristic; and wherein the shapable radiopaque coil with the shape-retention characteristic has a bending stiffness that is at least 10% of a bending stiffness of the catheter.

Other and further aspects and features will be evident from reading the following detailed description.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only exemplary embodiments and are not therefore to be considered limiting in the scope of the claims.

FIG. 1 illustrates the guidewire having a radiopaque coil.

FIG. 2 illustrates a variation of the guidewire of FIG. 1, particularly showing the guidewire having a malleable structure.

FIG. 3 illustrates a variation of the guidewire of FIG. 1, particularly showing the guidewire having a radiopaque coil that is implemented as a part of a sleeve.

FIG. 4 illustrates a technique of securing a radiopaque coil to shaft of the guidewire.

FIG. 5 illustrates a chart showing stress-strain curves for different respective materials.

FIG. 6 illustrates radiopaque characteristics for different respective materials.

FIG. 7 illustrates a catheter having a radiopaque coil.

FIG. 8 illustrates a variation of the catheter of FIG. 7.

FIG. 9 illustrates another variation of the catheter of FIG. 7.

### DETAILED DESCRIPTION

Various embodiments are described hereinafter with reference to the figures. It should be noted that the figures are not drawn to scale and that elements of similar structures or functions are represented by the same reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

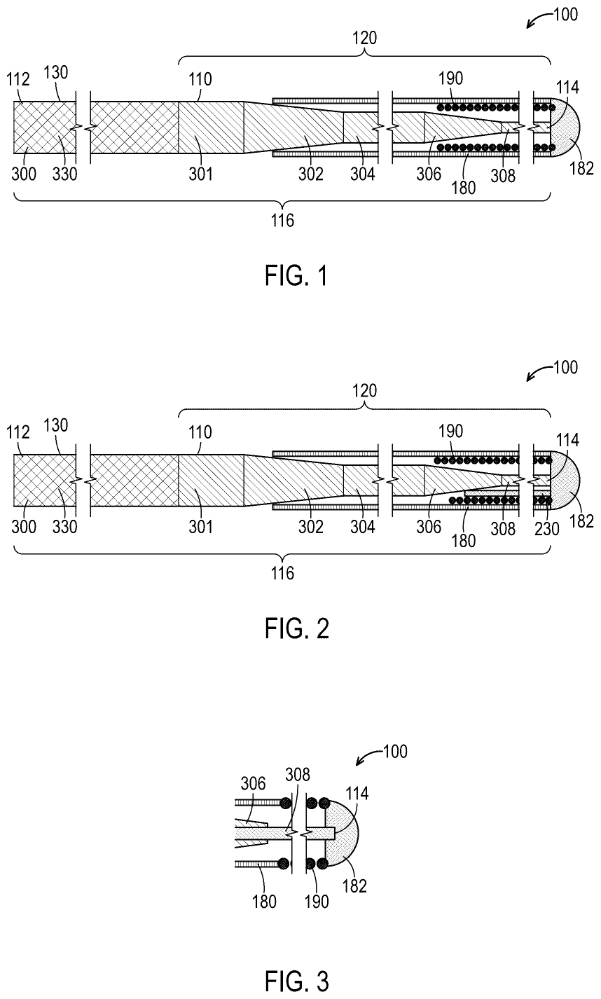

FIG. 1 illustrates a guidewire 100 in accordance with some embodiments. The guidewire 100 includes a shaft 110 having a proximal end 112, a distal end 114, and a body 116 extending from the proximal end 112 to the distal end 114. The body 116 of the shaft 110 comprises a distal segment 120 having a plurality of different cross sections along a length of the distal segment 120. At least an outer part 130 of the body 116 that is proximal to the distal segment 120 is made of a material having a shear modulus of at least 13000 ksi. By means of non-limiting examples, the material of the outer part 130 of the body is Molybdenum Rhenium alloy or Tungsten Rhenium alloy.

In other embodiments, the outer part 130 of the body 116 may be made of a material having a shear modulus that is less than 13000 ksi. Also, in other cases, the material of the outer part 130 may be different from Molybdenum Rhenium alloy or Tungsten Rhenium alloy.

In the illustrated embodiments, the shaft 110 includes a proximal segment 300 made of a first material, and the distal segment 120 is made from a second material that is different from the first material. In some embodiments, the proximal segment 300 may be made from Molybdenum Rhenium alloy or Tungsten Rhenium alloy. Also, in some embodiments, the distal segment 120 may be made from Nitinol, stainless steel, or Cobalt-Chromium alloy (e.g., MP35N alloy).

The proximal segment 300 may be attached to the distal segment 120 via an adhesive, weld, mechanical connector, or fusion.

In other embodiments, the proximal segment 300 and the distal segment 120 may be made from the same material. In one implementation, the segment 300 and the distal segment 120 may have a unity configuration and may be made from a same material.

As shown in the figure, the distal segment 120 includes a first part 301, a second part 302, a third part 304, a fourth part 306, and a fifth part 308. The fifth part 308 has a cross sectional dimension that is smaller compared to a cross sectional dimension of the third part 304, and the smaller cross sectional dimension of the fifth part 308 transitions into the larger cross section dimension of the third part 304 via the fourth (intermediate) part 306. Similarly, the third part 304 has a cross sectional dimension that is smaller compared to a cross sectional dimension of the first part 301, and the smaller cross sectional dimension of the third part 304 transitions into the larger cross section dimension of the first part 301 via the second (intermediate) part 302. Such configuration is advantageous because it provides progressively softer sections in the proximal-to-distal direction. As a result, the distal segment 120 is softer compared to the remaining part of the body 116, with the distal part 308 providing the softest section, and may flex or bend more easily. In other embodiments, the distal segment 120 may comprise more parts or fewer parts than those described above. In further embodiments, the segment 120 may have a same cross-sectional dimension and/or cross-sectional shape along its entire length.

As shown in the figure, an inner part 330 of the body 116 that is proximal to the segment 120 and the outer part 300 of the body 116 that is proximal to the segment 120 are made from the same material (as shown by the shaded cross section). In one implementation, the outer part 300 and the inner part 330 of the body 116 are made from a same piece of raw material (e.g., Molybdenum Rhenium alloy, Tungsten Rhenium alloy, etc.), so that they have an unity configuration. In other embodiments, the outer part 300 and the inner part 330 may be made from other materials. Also, in further embodiments, the outer part 300 and the inner part 300 may be made from different respective materials.

In some embodiments, the part 308 of the segment 120 may be compressed to form an elongated cross-sectional shape for the part 308. For example, in one implementation, the part 308 may have a circular cross-sectional shape, and the part 308 of the segment 120 may be compressed into a planar structure having an elongated cross sectional shape (e.g., substantially, rectangular shape), or any of other non-circular cross sectional shapes. This feature is advantageous because it provides a bias in the direction of bending for the part 308. Also, the elongated cross-sectional shape is advantageous because it allows the part 308 of the segment 120 to be flexible enough for bending in one plane while having enough cross-sectional area to achieve a desirable tensile strength. The compressing of the part 308 may be achieved by compressing (e.g., stamping, rolling, etc.) the part 308 in some embodiments. In other embodiments, the part 308 of the segment 120 may not be compressed, and/or may have a non-elongated cross sectional shape. For example, in other embodiments, the part 308 of the segment 120 may have a circular cross sectional shape, a square cross sectional shape, etc.

In the illustrated embodiments, the guidewire 100 also includes a sleeve 180 disposed around the distal end 114 of the shaft 110. As shown in the figure, the sleeve 180 has a blunt tip 182, or is coupled to the blunt tip 182. The distal end 114 of the shaft 110 is also coupled to the blunt tip 182. The sleeve 180 may be any tubular member, and may be made from any materials, such as metal, polymer, etc. In some embodiments, the sleeve 180 may be made from Nitinol. The sleeve 180 may have a plurality of slots and/or openings to increase a flexibility of the sleeve 180. By means of non-limiting examples, the sleeve 180 may be implemented using slotted hypotube, coiled sleeve, tungsten-loaded polymer sleeve, or a combination of the foregoing.

In one specific implementation, the proximal segment 300 is made from Molybdenum Rhenium alloy, and the distal segment 120 is made from Nitinol. In another specific implementation, proximal segment 300 is made from Molybdenum Rhenium alloy, and the distal segment 120 is made from stainless steel, or Cobalt-Chromium alloy (e.g., MP35N alloy). In either implementation, the distal segment 120 provides kink resistance and a soft distal end for the guidewire 100, while the proximal segment 300 provides a desired pushability and a desired torqueability. The distal segment 120 may be shapeable during use, and provides desirable shape retention capability (e.g., with or without the aid of the malleable structure 230). Also, because of its relatively high shear modulus, the Molybdenum Rhenium alloy proximal segment 300 provides a desirable torqueability. Furthermore, because of the axial stiffness of the Molybdenum Rhenium alloy segment 300, the guidewire 100 also has a desirable pushability. In some cases, the proximal segment 300 and the distal segment 120 may be made from one or more materials having one or more respective elastic modulus to provide a desirable bending stiffness for the device.

As shown in FIG. 1, the guidewire 100 also includes a coil 190 disposed inside the sleeve 180. As shown in the figure, one end of the coil 190 is coupled (e.g., secured directly or indirectly, extending from, etc.) to the tip 182. In other embodiments, the proximal end of the coil 190 may be coupled (e.g., secured directly or indirectly, extending from, etc.) to distal segment 120 (e.g., to any location that is proximal to the part 308, such as to the part 306, the part 304, or the part 302, etc.). The securing may be accomplished using an adhesive, welding, mechanical connector, fusion, etc. In other embodiments, the coil 190 may also be coupled to the wall of the sleeve 180. In further embodiments, at least a part of the sleeve 180 may be formed by the coil 190 (FIG. 3). In such cases, the coil 190 has a cross sectional dimension that corresponds with (e.g., is the same as) a cross sectional dimension of the sleeve 180. In other embodiments, the coil 190 may form an entirety of the sleeve 180.

In the illustrated embodiments, the coil 190 is made from Molybdenum Rhenium (MoRe). This allows the coil 190 to function as a radiopaque coil during a medical procedure. The coil 190 made from MoRe also provides the coil 190 with a desirable stiffness and shape retention characteristic. The shape retention characteristic of the coil 190 assists the distal portion of the guidewire 100 to maintain a bent shape after the distal portion of the guidewire 100 has been bent during the medical procedure.

In some cases, the wire making up the coil 190 may have a cross-sectional dimension (e.g., diameter) that is anywhere from 0.001 inch to 0.005 inch. Also, the wire making up the coil 190 may have a length (unwind length) that is anywhere from 5 cm to 150 cm. In other cases, the wire making up the coil 190 may have a cross-sectional dimension that is less than 0.001 in or higher than 0.005 inch, and/or may have an unwind length that is higher than 20 cm.

In some cases, the coil 190 may have a length anywhere from 1 cm to 10 cm. Also, the coil 190 may have an outer diameter that is anywhere from 0.004 inch to 0.05 inch, and more preferably anywhere from 0.006 inch to 0.02 inch, and more preferably anywhere from 0.008 inch to 0.01 inch.

In some cases, the loops of the coil 190 may contact respective adjacent (neighboring) loops. In other cases, the coil 190 has an open pitch, wherein the spacing between adjacent loops of the coil 190 may be anywhere between 0.0001 inch to 0.002 inch, or higher.

It should be noted that the coil 190 is advantageous because it is better at retaining shape than cylindrical or flat solid sections. This is due to the Bauschinger effect—when the coil 190 is shaped, the strains are primarily in shear and are evenly distributed along the entire length of the wire forming the coil 190. The Bauschinger effect becomes more pronounced with higher levels of cold work. Since the coil 190 receives a relatively small amount of cold work during shaping (compared to the stamped tip for example), the shape retention of the coil 190 is superior. In addition, in some cases, the stiffness of the coil 190 may be a small percentage of the overall distal tip stiffness. Accordingly, the coil 190 shapes well, and it doesn't contribute as much to the system behavior. In some cases, the coil 190 may be made stiffer, while the other components of the guidewire 100 may be made softer, thereby achieving better tip-shape-retention (TSR). MoRe has a modulus of shear and elasticity at least twice that of Pt alloys.

The stiffness (e.g., bending stiffness) of the coil 190 may be adjusted by incorporating different amount of Rhenium (Re) in the alloy MoRe. By means of non-limiting examples, the MoRe alloy may have 47%, 47.5%, 41%, 25% of Rhenium by weight. In some cases, the amount of Rhenium in the MoRe alloy is at least 15%, and preferably at least 20%, and more preferably at least 30% (e.g., anywhere from 30%-60%) by weight.

One technique of making the coil 190 is to wind a wire or elongated element made from MoRe (or any of other desirable materials described herein) onto a mandrel. The cross-section shape of the elongated element (e.g. wire) making up the coil 190 could be round, rectangular, elliptical or any other shape, and the resulting coil 190 could be modified by removing or adding material after winding, or axially stretching or compressing. Another technique of making the coil 190 is to make a coil cut (e.g., laser-cut) through a wall of a tube made from MoRe or any of other desirable materials described herein. Thus, as used in this specification, the term “coil” is not limited to a wire wound into a coil configuration. Optionally, the coil 190 may be annealed after winding the wire to mitigate the cold work induced during the winding. Also, optionally, one or more hardening techniques, such as (configuring wire drawing parameters, thermal treatments, shot peening, etc.) may be employed to process the coil 190 in order to achieve a desirable stiffness for the coil 190.

In the illustrated embodiments of FIG. 1, the distal part 308 of the segment 120 is malleable. Thus, the distal part 308 of the segment 120 is bendable to form a bent shape. The distal part 308 is made from a material that allows it to retain the bent shape after the distal part 308 of the segment 120 is bent. In such cases, the coil 190 is also malleable and bendable so that the coil 190 and the distal part 308 of the segment 120 can be bent together. The coil 190 also retains its bent shape so that both the distal part 308 of the segment 120 and the coil 190 can together retain the bent shape for the guidewire 100. In some embodiments, the distal part 308 of the segment 120 alone does not provide sufficient bending stiffness. In such cases, the coil 190 can provide additional bending stiffness. It is desirable for the coil 190 to provide a significant portion (e.g., at least 10%, at least 20%, at least 30%, at least 40%, at least 50%, at least 60%, at least 70%, or higher, etc.) of the total bending stiffness so that its shape retention contributes significantly to the system. The stiffness of the coil 190 can be tuned by varying the coil pitch, coil wire diameter, coil wire modulus of elasticity, material composition, or any combination of the foregoing. It should be noted that achieving a desired bending stiffness for the coil 190 is not easy because the wire of the coil 190 undergoes torsion when the coil 190 is bent. By utilizing the materials and techniques described herein to make the coil 190, a desired bending stiffness of the coil 190 can be achieved.

In other embodiments, the distal part 308 may be made from a material that does not have sufficient shape retention capability. In such cases, the coil 190 will provide the shape retention characteristic for the guidewire 100. Thus, the coil 190 may either (1) assist the distal part 308 to provide a desired shape retention characteristic for the guidewire 100, or (2) provide at least a majority (e.g., all) of the shape retention characteristic for the guidewire 100.

In further embodiments, both the distal part 308 and the coil 190 together may not provide sufficient shape retention ability for the guidewire 100. In such cases, the guidewire 100 may further include a malleable structure 230 (e.g., a flat plate, a wire, a shaping ribbon, etc.) attached to the blunt tip 182 (FIG. 2). The malleable structure 230 may be inside the sleeve 180. During use, the malleable structure 230 is bendable to form a bent shape and is configured to retain the bent shape after the malleable structure 230 is bent.

It should be noted that the guidewire 100 is not limited to the examples of FIGS. 1-2, and that the guidewire 100 may have other configurations in other embodiments. In other embodiments, the guidewire 100 may include more than two segments. For example, in other embodiments, the guidewire 100 may include three or more segments that are connected in series along a longitudinal axis of the guidewire 100. Also, in some embodiments, at least one of the segments of the guidewire 100 may have multiple layers, or a single layer.

The guidewire 100 is advantageous because it provides an optimal combination of shapeability, shape retention capability, and torqueability. Due to the use of MoRe to construct the coil 190, the resulting guidewire 100 may have a small profile. Accordingly, the guidewire 100 may be used to access smaller blood vessels, such as distal blood vessels in a brain, thereby reaching more aneurysms that cannot be accessed before. The coil 190 made from MoRe allows the distal portion of the guidewire 100 to be shapeable during use, and provides desirable shape retention capability (without the aid of a malleable structure). However, in other embodiments, the guidewire 100 may optionally further include a malleable structure to enhance the shape retention capability, as similarly discussed. Also, the distal portion of the guidewire 100 provides a desirable torqueability, kink resistance, and a soft distal end for the guidewire 100.

The material for making the coil 190 is not limited to MoRe, and the coil 190 may be made from other materials in other cases. For example, in other cases, the coil 190 may be made from MoRe in combination with other alloying element(s), such as Zr and/or Hf. In further cases, the coil 190 may be made from Tungsten Rhenium or Tungsten with other alloying element(s), such as Zr and/or Hf. Any of these materials may allow the radiopaque coil 190 to achieve the desired bending stiffness. Other materials that are different from the above examples may also be used to make the coil 190 in other cases.

In some cases, the elastic modulus of the material that allows the radiopaque coil 190 to achieve a desired bending stiffness is at least 3E7 psi, and preferably 4E7 or higher. A desirable bending stiffness for the coil 190 is at least 10%, at least 20%, at least 30%, at least 40%, at least 50%, at least 60%, at least 70%, or higher, of the bending stiffness of the guidewire 100.

In addition, in some cases, the material of the coil 190 may exhibit x-ray attenuation that is similar to or greater than that of Pd (having equal thickness as the material), at x-ray energy levels that are suitable for imaging. The x-ray attenuation exhibited by the material of the coil 190 may be considered as being “similar” to that of Pd of equal thickness if the x-ray attenuation Aₘ exhibited by the material of the coil 190 does not vary from the x-ray attenuation A_{pd} exhibited by Pd by more than 20% (i.e., 0.8A_{pd}<=Aₘ<=1.2A_{pd}).

Also, it should be noted that the materials for making the shaft 110 of the guidewire 100 should not be limited to the examples described, and that the shaft 110 may be made from other materials in other embodiments. For example, in other embodiments, the shaft 110 of the guidewire 100 may be made from other materials, as long as a desired torqueability is achieved. In other embodiments, the shaft 110 may be made from any material having a Young's Modulus (under annealed condition) of at least 6000 ksi, or more preferably at least 30000 ksi, or even more preferably at least 40000 ksi. Also, in other embodiments, the shaft 110 may be made from any material having an ultimate tensile strength of at least 100 ksi, and more preferably at least 200 ksi, and even more preferably at least 300 ksi. By means of non-limiting examples, specific materials that may be used include, but are not limited to, Mo-47.5Re, W-25Re, SS304, etc.

In some cases, MoRe may have a tensile strength that is almost 1 M ksi. This may be acceptable for use to construct the shaft 110 of the guidewire 100. However, for the coil 190, it would be desirable to utilize a version of MoRe, or another material, with lower tensile strength (and thus, lower yield strength) to achieve shapeability. In some cases, the ultimate tensile strength (UTS) may be in the range of 200 to 400 kpsi. In some cases, the UTS and yield strength of the coil 190 may be reduced by inducing lower cold work during wire drawing. Subsequent heat treatment (annealing) may also be used to reduce the UTS for the coil 190.

In addition, it should be noted that the shaft 110 of the guidewire 100 may have different dimensions in different embodiments. For example, in some embodiments, the shaft 110 of the guidewire 100 may have a total length that is anywhere from 50 inches to 100 inches, such as a length that is anywhere from 70 inches to 90 inches. Also, in some embodiments, the distal segment 120 may have a length that is 5 inches to 30 inches, such as a length that is anywhere from 10 inches to 25 inches, or a length that is anywhere from 12 inches to 20 inches. Furthermore, in some embodiments, the distal part 308 may have a length that is anywhere from 0.3 inch to 1 inch, such as a length that is anywhere from 0.5 inch to 0.8 inch. In some embodiments in which the distal part 308 is stamped, the stamped distal part may have a portion with a constant width, wherein the portion may have a longitudinal length of at least 0.3 inch, such as at least 0.4 inch. In addition, in some embodiments, the distal segment 120 may have a cross sectional dimension (e.g., diameter) that is anywhere from 0.00157 inch to 0.0197 inch (0.04 mm to 0.5 mm) and the distal part 308 may have a cross sectional dimension (e.g., diameter) that is anywhere from 0.000157 inch to 0.00394 inch (0.004 mm to 0.1 mm). In other embodiments, the distal segment 120 and/or the distal part 308 may have dimensions that are different from those mentioned above.

Furthermore, the number of different cross sections along the length of the distal segment 120 is not limited to the examples described previously. In other embodiments, the number of different cross sections along the length of the distal segment 120 may be more, or fewer, than the ones described herein.

In addition, in one or more embodiments described herein, a part of the segment 120 may have a flat portion, as similarly discussed. For example, the part 308 (in the embodiments of FIG. 1 or 2), may be compressed (e.g., stamped, rolled, etc.) to create a flat portion. In some embodiments, the flat portion of the segment 120 may include one or more openings extending through a thickness of the flat portion. This feature is advantageous because it may assist the guidewire 100 in achieving a soft distal end without compromising other performance, such as shapeability, tensile strength, and/or shape retention of the guidewire 100.

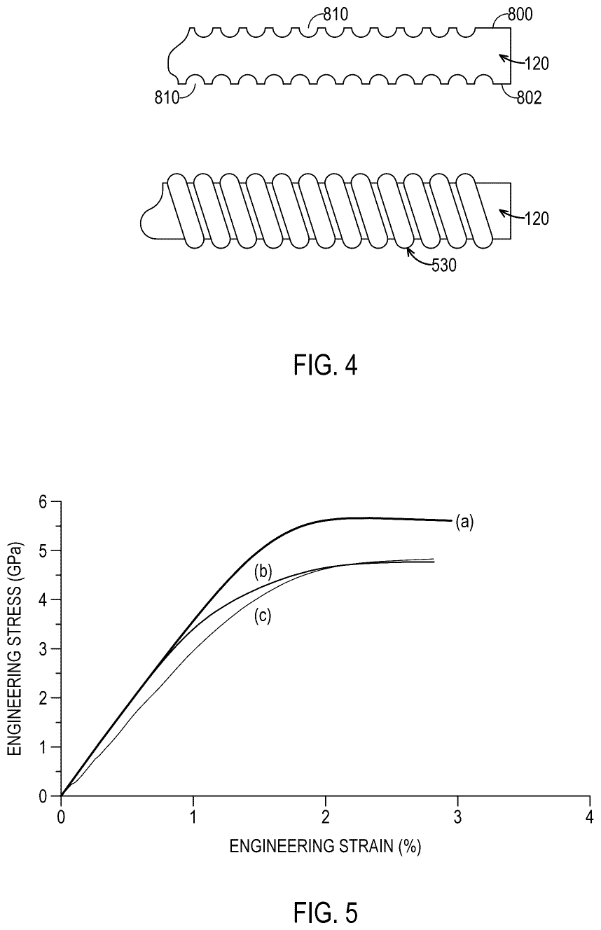

Furthermore, it should be noted that the manner in which the coil 190 is secured to the shaft 110 is not limited to the examples described. In other embodiments, the coil 190 may be secured to the shaft in 110 other manners. For example, as shown in FIG. 4, in some embodiments, the coil 190 of the guidewire 100 may be a radiopaque coil 190 surrounding at least a part of a segment 120 of the shaft 110 of the guidewire 100. The part of the segment 120 has opposite sides 800, 802 with indentations 810 along each of the opposite sides for allowing the radiopaque coil 190 to be screwed over the part of the segment 120. In some embodiments, the part of the segment 120 surrounded by the radiopaque coil 190 may be a flat portion. In such cases, the indentations 810 may be grooves extending through the thickness of the flat portion. The indentations 810 may be implemented as cutouts in some embodiments. In other embodiments, the part of the segment 120 surrounded by the coil 190 may be another part of the segment 120 that is proximal to the flat portion 308. In further embodiments, the segment 120 surrounded by the radiopaque coil 190 may not be any flat portion. Instead, the segment 120 surrounded by the radiopaque coil 190 may be an un-flattened portion of a core wire or shaft. The technique shown in FIG. 4 for attaching the coil 190 to the segment 120 is advantageous because it provides mechanical interaction between the segment 120 and the coil 190, and it also save space inside an outer distal sleeve of the guidewire 100. It is also advantageous because the joining strength is attributable to the mechanical interface between the segment 120 and the coil 190, rather than an adhesive interface. In some cases, the segment 120 may be manufactured by laser profiling a stamped member, which allows the coil 190 to thread on.

In any of the embodiments described herein, the guidewire 100 may be provided as a part of a medical device. For example, a medical device may include a catheter, and the guidewire 100, wherein the catheter includes a lumen for accommodating the guidewire 100. By means of non-limiting examples, the medical device may be a microcatheter, a balloon catheter, a stent delivery catheter, a catheter for removing blockage in a vessel, a delivery catheter for the guidewire 100, etc.

In one method of use of the guidewire 100, a doctor first bends the distal segment of the guidewire 100 into a desired shape, depending on the geometry of the anatomy that the guidewire 100 will access. For example, the distal segment of the guidewire 100 may be bent to have a L shape, a C shape, a U shape, a S shape, a shape with two or more curves in different planes, etc. The guidewire 100 is then placed in a delivery catheter. Then, an incision is made at a skin of a patient. The delivery catheter with the guidewire 100 therein is inserted through the incision, and into a blood vessel in the patient. The delivery catheter and the guidewire 100 may be advanced distally until the distal end of the guidewire 100 and/or the delivery catheter reaches a target site. The target site may be anywhere in the patient's body, such as a blood vessel in a limb, in a torso, in a neck, in a head, etc. The delivery catheter houses the guidewire 100 as the delivery catheter is advanced distally. When the delivery catheter reaches a location in the patient that requires the bent shape of the distal segment of the guidewire 100 to access, at least a part of the distal segment may be deployed out of the delivery catheter to let the distal segment assumes its bent shape. The guidewire 100 may be torqued to direct the bent shape in the direction of desired access. The bent shape of the distal segment of the guidewire 100 steers the guidewire 100 into a desired direction, thereby allowing the guidewire 100 and the delivery catheter to be advanced distally into a desired passage. The guidewire 100 described herein is advantageous because it allows a bent shape of the distal segment of the guidewire 100 to be retained, so that the bent shape will not return back to the pre-bent configuration even after the distal segment has traversed different paths in a vessel with different curvatures (or even after the bent distal segment has been placed in a tube, such as a delivery tube). The guidewire 100 is also advantageous because it allows the doctor to effectively torque the guidewire 100 due to the enhanced torqueability of the guidewire 100, and allows the doctor to push the guidewire 100 distally inside the patient without kinking.

Embodiments of the guidewire 100 described herein have desired torqueability, desired shape retention capability, desired pushability, or any combination of the foregoing. In some embodiments, a desired torqueability is considered to be achieved by the guidewire 100 if a twisting or torqueing motion applied at a proximal end about a longitudinal axis of the guidewire 100 to turn the proximal end of the guidewire 100 (or shaft 110) by an angle P will result in a turning of the distal end of the guidewire 100 by an angle D that is at least 80% of P, or more preferably at least 90% of P, or even more preferably at least 95% of P (e.g., 100% of P, which means that the distal end of the guidewire 100 has 1:1 response with respect to a torque applied at the proximal end of the guidewire 100). Also, in some embodiments, a desired shape retention capability is considered to be achieved by the guidewire 100 if the bent segment with curvature can retain at least 70% of the curvature, or more preferably at least 80% of the curvature, and even more preferably at least 90% of the curvature, after the bent segment is placed in a tube and is pushed back out from the tube. Also, in some embodiments, a desired pushability may be achieved if the guidewire 100 does not kink while being advanced inside a vessel.

It should be noted that using MoRe to make the radiopaque coil 190 is advantageous. MoRe has an elastic modulus that is 2.5-3 times greater than that of Platinum or Platinum alloys (see FIG. 5). In the figure, curve (a) is a stress-strain curve for a round wire having a diameter of 25 um made from Mo41 Re, curve (b) is a stress-strain curve for a round wire having a diameter of 25 um made from W25Re, and curve (c) is a stress-strain curve for a round wire having a diameter of 25 um made from Mo47Re. The guidewire 100 may be required to have a low profile (e.g., diameter), which limits the size of wire that can be used to make radiopaque coil 190 for the guidewire 100, limits how stiff such coil 190 can be, and limits how useful the coil 190 can be used to assist the guidewire 100 to retain the distal bent shape. Making radiopaque coil using the MoRe material with higher stiffness, or other materials described herein, improves shape retention for the guidewire over traditional materials.

Also, in some embodiments, through proper selection of heat treating parameters, the yield strain of the material (e.g., MoRe) in the radiopaque coil 190 may be tuned so that the coil 190 can have a curvature that corresponds with the curvature of a core (e.g., the shaft 110) during a shaping process. Alternatively or additionally, the material in the radiopaque coil 190 may also be tuned so that the shapeability and/or the shape retention characteristic of the coil 190 (or of the guidewire/catheter) may be improved (e.g., optimized). In some cases, heat treatment after coil winding may help eliminate the cold work hardening that can take place during the coil winding.

In addition, making the coil 190 using MoRe or other materials described herein is advantageous because it may have a better shape retention ability than a similarly shaped corewire ribbon due to a reduced impact of the Bauschinger effect. The Bauschinger effect is the tendency for a plastically-deformed metal to plastically deform at a lower stress level, in the direction opposite that of the original deformation. The degree of this effect is proportional to the degree of the initial plastic deformation. Since the deformation of the coil (in torsion) is spread over a greater length of the coil wire to get the same gross curvature in a coil versus a corewire ribbon, the impact of the Bauschinger effect should be reduced in the coil structure.

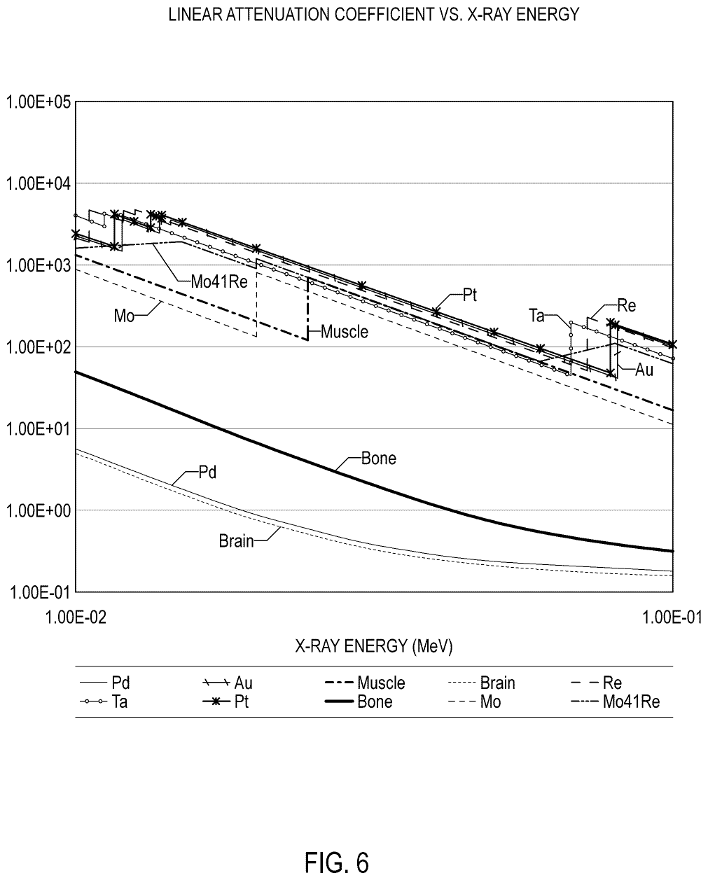

Furthermore, making the coil 190 using the materials described herein (e.g., MoRe) is also advantageous because it provides sufficient radiopacity for application in medical procedures. The attenuation of an alloy is the sum of the individual coefficients of the elements, each multiplied by the weight fraction present in the alloy. As shown in FIG. 6, in the relevant x-ray energy range, Mo41 Re has attenuation close to that of Pt and Au, and is slightly better than that of Pd and Ta, which are sometimes used in radiopaque elements.

The coil made from the material described herein (e.g, MoRe, WRe, etc.) is not limited for application in guidewire, and made be applied for other types of medical devices. In other embodiments, the coil made from MoRe, WRe, etc., may be a part of a push wire or a delivery wire. In further embodiments, the coil made from MoRe, WRe, etc., may be a part of a catheter, such as a delivery catheter, a diagnostic catheter, a treatment catheter, a balloon catheter, etc.

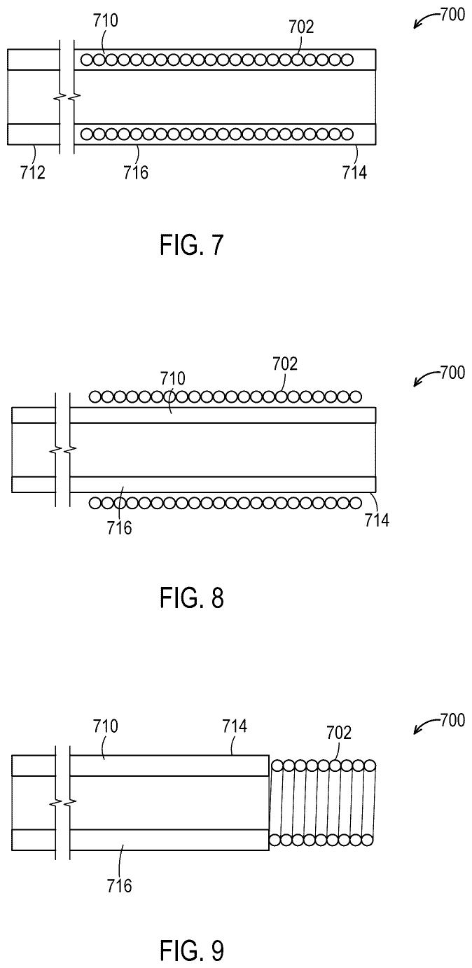

FIG. 7 illustrates a catheter 700 having a coil 702 that is made from MoRe, WRe, or another material described herein. The catheter 700 may be a delivery catheter configured to deliver an item, such as drug, medicine, agent, implant (e.g., vaso-occlusive device, stent, etc.), a biopsy device, a treatment device, etc. In other embodiments, the catheter 700 may be a delivery catheter configured to obtain a biopsy or tissue sample from the patient, and deliver it to a container outside the patient. In some embodiments, the catheter 700 may optionally further include an inflatable balloon.

As shown in the figure, the catheter 700 includes a tubular member 710 having a proximal end 712, a distal end 714, and a body 716 extending from the proximal end 712 to the distal end 714. The coil 702 is a radiopaque coil made from MoRe, WRe, or another materials described herein, and is coupled to the tubular member 710. The radiopaque coil 702 has a stiffness that is greater than a coil having a same size and shape as those of the radiopaque coil, but is made from platinum or platinum alloys. Also, at least a part of the radiopaque coil 702 is configured to have a curvature that corresponds with a curvature of a part of the body 716 of the tubular member 710.

In some embodiments, the radiopaque coil 702 is optionally shapable and has a shape-retention characteristic.

In the above embodiments, the coil 702 is located within a wall of the tubular member 710. In other embodiments, the coil 702 may be disposed outside the tubular member 710 surrounding the tubular member 710 (FIG. 8). In further embodiments, the coil 702 may be disposed distal to the tubular member 710 (FIG. 9). In such cases, the coil 702 may be secured to the tubular member 710 by an adhesive, and/or by a sleeve that engages with both the coil 702 and the tubular member 710. The sleeve may be disposed on an inside surface of the tubular member 710 and an inside surface of the coil 702. Alternatively, the sleeve may be disposed on an outer surface of the tubular member 710 and an outer surface of the coil 702. In further embodiments, at least a part of the tubular member 710 and at least a part of the coil 702 may be disposed inside the sleeve.

The following items are exemplary features of embodiments described herein. Each item may be an embodiment itself or may be a part of an embodiment. One or more items described below may be combined with other item(s) in an embodiment.

Item 1: A medical device includes: an elongate member having a proximal end, a distal end, and a body extending from the proximal end to the distal end; a blunt tip at or coupled to the distal end of the elongate member; and a radiopaque coil coupled to the elongate member; wherein the radiopaque coil is shapable and has a shape-retention characteristic, and wherein the shapable radiopaque coil with the shape-retention characteristic has a bending stiffness that is at least 10% of a bending stiffness of the medical device.

Item 2: The bending stiffness of the shapable radiopaque coil is at least 20% of the bending stiffness of the medical device.

Item 3: The shapable radiopaque coil with the shape-retention characteristic is made from a material having an elastic modulus that is at least 3E7 psi.

Item 4: The shapable radiopaque coil with the shape-retention characteristic is made from Molybdenum Rhenium (MoRe), or another Molybdenum alloy.

Item 5: The shapable radiopaque coil with the shape-retention characteristic is made from Tungsten, Tungsten Rhenium (WRe), or another Tungsten alloy.

Item 6: The radiopaque coil is configured to assist retention of a shape for the medical device.

Item 7: The bending stiffness of the radiopaque coil is greater than a bending stiffness of a coil having a same size and shape as those of the radiopaque coil, but is made from platinum or platinum alloys.

Item 8: The radiopaque coil has been heat-treated to optimize a shape-ability and the shape retention characteristic of the radiopaque coil.

Item 9: The medical device is a guidewire.

Item 10: The elongate member is a shaft, and wherein the radiopaque coil surrounds at least a part of the shaft.

Item 11: The medical device further includes a sleeve disposed around the radiopaque coil.

Item 12: The shaft comprises a flat portion.

Item 13: The shaft comprises a tapering portion that is proximal the flat portion.

Item 14: The shaft comprises an additional flat portion or a cylinder portion that is proximal the tapering portion.

Item 15: The medical device is a catheter.

Item 16: The elongate member is a tubular member, and wherein the radiopaque coil is coupled to the tubular member.

Item 17: The radiopaque coil is disposed circumferentially around the tubular member, or is distal to the tubular member.

Item 18: The blunt tip comprises a rectilinear surface at the distal end of the elongate member.

Item 19: A guidewire includes: a shaft having a proximal end, a distal end, and a body extending from the proximal end to the distal end; a blunt tip coupled to the distal end of the shaft; and a radiopaque coil disposed around at least a part of the shaft; wherein the radiopaque coil is shapable and has a shape-retention characteristic; and wherein the shapable radiopaque coil with the shape-retention characteristic has a bending stiffness that is at least 10% of a bending stiffness of the guidewire.

Item 20: A catheter includes: a tubular member having a proximal end, a distal end, and a body extending from the proximal end to the distal end; and a radiopaque coil coupled to the tubular member; wherein the radiopaque coil is shapable and has a shape-retention characteristic; and wherein the shapable radiopaque coil with the shape-retention characteristic has a bending stiffness that is at least 10% of a bending stiffness of the catheter.

Although particular embodiments have been shown and described, it will be understood that it is not intended to limit the claimed inventions to the preferred embodiments, and it will be obvious to those skilled in the art that various changes and modifications may be made without department from the spirit and scope of the claimed inventions. The specification and drawings are, accordingly, to be regarded in an illustrative rather than restrictive sense. The claimed inventions are intended to cover alternatives, modifications, and equivalents.

1. A medical device, comprising:
an elongate member having a proximal end, a distal end, and a body extending from the proximal end to the distal end;
a blunt tip at or coupled to the distal end of the elongate member; and
a radiopaque coil coupled to the elongate member;
wherein the radiopaque coil is shapable and provides a shape-retention characteristic for the medical device.

2. The medical device of claim 1, wherein the bending stiffness of the shapable radiopaque coil is at least 20% of the bending stiffness of the medical device.

3. The medical device of claim 1, wherein the shapable radiopaque coil with the shape-retention characteristic is made from a material having an elastic modulus that is at least 3E7 psi.

4. The medical device of claim 1, wherein the shapable radiopaque coil with the shape-retention characteristic is made from Molybdenum Rhenium (MoRe), or another Molybdenum alloy.

5. The medical device of claim 1, wherein the shapable radiopaque coil with the shape-retention characteristic is made from Tungsten, Tungsten Rhenium (WRe), or another Tungsten alloy.

6. The medical device of claim 1, wherein the radiopaque coil is configured to assist retention of a shape for the medical device.

7. The medical device of claim 1, wherein the bending stiffness of the radiopaque coil is greater than a bending stiffness of a coil having a same size and shape as those of the radiopaque coil, but is made from platinum or platinum alloys.

8. The medical device of claim 1, wherein the radiopaque coil has been heat-treated to optimize a shape-ability and the shape retention characteristic of the radiopaque coil.

9. The medical device of claim 1, wherein the medical device is a guidewire.

10. The medical device of claim 9, wherein the elongate member is a shaft, and wherein the radiopaque coil surrounds at least a part of the shaft.

11. The medical device of claim 10, further comprising a sleeve disposed around the radiopaque coil.

12. The medical device of claim 10, wherein the shaft comprises a flat portion.

13. The medical device of claim 12, wherein the shaft comprises a tapering portion that is proximal the flat portion.

14. The medical device of claim 13, wherein the shaft comprises an additional flat portion or a cylinder portion that is proximal the tapering portion.

15. The medical device of claim 1, wherein the medical device is a catheter.

16. The medical device of claim 15, wherein the elongate member is a tubular member, and wherein the radiopaque coil is coupled to the tubular member.

17. The medical device of claim 16, wherein the radiopaque coil is disposed circumferentially around the tubular member, or is distal to the tubular member.

18. The medical device of claim 15, wherein the blunt tip comprises a rectilinear surface at the distal end of the elongate member.

19. A guidewire, comprising:
a shaft having a proximal end, a distal end, and a body extending from the proximal end to the distal end;
a blunt tip coupled to the distal end of the shaft; and
a radiopaque coil disposed around at least a part of the shaft;
wherein the radiopaque coil is shapable and has a shape-retention characteristic; and
wherein the shapable radiopaque coil with the shape-retention characteristic has a bending stiffness that is at least 10% of a bending stiffness of the guidewire.

20. A catheter, comprising:
a tubular member having a proximal end, a distal end, and a body extending from the proximal end to the distal end; and
a radiopaque coil coupled to the tubular member;
wherein the radiopaque coil is shapable and has a shape-retention characteristic; and
wherein the shapable radiopaque coil with the shape-retention characteristic has a bending stiffness that is at least 10% of a bending stiffness of the catheter.
